(19) United States
(12) Patent Application Publication
(10) Pub. No.: US 2019/0343670 A1
(43) Pub. Date: Nov. 14, 2019

(54) Wearable Mobility Assisting Apparatus

(71) Applicant: Elite Athlete Products, Inc., San Diego, CA (US)

(72) Inventors: Cathy Ruprecht, San Diego, CA (US); Julie Sanford, San Diego, CA (US); Kimberly Spranger, San Diego, CA (US); Lynette Vanwelsenaers, San Diego, CA (US)

(21) Appl. No.: 16/408,366

(22) Filed: May 9, 2019

Related U.S. Application Data
(60) Provisional application No. 62/669,346, filed on May 9, 2018.

(51) Int. Cl. A61F 5/01; A61H 3/00; A63B 21/04

(52) U.S. Cl. CPC A61F 5/0113; A61H 3/00; A61H 2201/165; A61H 2003/007; A61H 2201/1642; A63B 21/0442

(57) ABSTRACT

A wearable mobility assisting device is provided having one or a plurality of elastic bands extending between anchors positioned on an anchor belt engaged to the body of the user at a first location and a limb belt engaged to the body of the user in a second position. The anchors may be slidably located to adjust the force vector or angle of the elastic bands between their engaged opposing ends. A shoulder harness may also be provided for additional stability of the device during use.

This application claims priority to U.S. Provisional Patent Application Ser. No. 62/669346 filed on May 9, 2018, and incorporated herein by this reference thereto, in its entirety.

### BACKGROUND OF THE INVENTION

### 1. Field of the Invention

The present invention relates generally to mobility and stability enhancing devices for humans. More particularly, it relates to a wearable user-configurable resistive assistance device for adaptable to impart a force from one or a plurality of vectors, for aid and treatment of multiple types of movement pattern disabilities and asymmetries.

### 2. Prior Art

In the U.S. and throughout the world, multiple types of movement pattern disabilities and asymmetries exist which are problematic for those suffering such. Such movement pattern disabilities and asymmetries for example may include muscular weakness, presence of tone, range of motion deficits, and lack of proprioceptive input. All such afflictions and the foundational problems causing them to contribute to ineffective and/or painful movement patterns for persons having to deal with them on a daily basis.

However, frequently the solution for these movement pattern disabilities and asymmetries is not as simple eliminating the underlying problem. Movement patterns in short time periods and especially after many years become deeply ingrained in the day to day movements and physical actions of people having them. Consequently, even when the foundational problems which may cause or contribute to the errant movement patterns are alleviated, the movement patterns do not change. Physical therapists and exercise specialists often use cuing for sufferers to try to change these patterns. Such cuing may be visual, verbal or manual. Conventionally, manual cues are provided by either physical touch or use of a device or tape.

Conventional cuing devices which are currently available on the market to address such foundational problems and/or provide cuing to the patient to improved movement patterns, are either configured in a specific manner where they only provide cues for one problem, or they are very complicated to use and expensive to obtain. For example, patent publication numbers 2008/02967661 and 2011/0218466 are rigid and unadaptable in their configurations, and they are complicated for the user to properly place them on their body. While such complicated and fixed-configuration devices may be effective in a research setting, due to their complicated fixed configuration, it takes a significant amount of time during a physical therapy session just to engage the devices to the body of the user or patient. Further, such prior art devices are fixed configurations and are not re-configurable to allow treatment of multiple different foundational problems.

Other devices like the “foot-up” which was patented in 2010 (U.S. Pat. No. 7,674,212B2) can only address the foot drop issue, and the “Triple Flex” which was patented in 2014 can only flex the hip, knee and ankle collectively.

Due to fixed configurations of such prior art devices and the inability to reconfigure them for multiple physical issues, and the fact that such prior art devices are hard to properly position on a patient, many health care professionals/exercise specialists do not use mobility enhancing devices in their practices. Instead, they work on retraining muscle movements and strengthening in less functional patterns.

In such retraining and strengthening sessions, due to the above noted prior art issues, therapists use mostly visual and verbal cuing. Manual cuing if attempted or employed, is limited to hand placement or affixing of tape to the patient. While such tape employment may have a place for treatment of smaller issues, for bigger, grosser imbalances, it is minimally effective.

The device and system herein are easily configurable to multiple configurations to be worn to address multiple foundational problems of different patients, by simple sliding of one or more band anchors to multiple positions, and engaging elastic bands. Using user-worn belts, the slidable anchors provide variable positioned mounts for the elastic and resistive bands, and can be easily positioned to treat a wide variety of foundational problems. The bands can be employed to impart force to a common point along multiple vectors at varying magnitudes, to provide customized support and/or treatment to a particular physical problem. In combination with varying the force provided by the elastic bands employed, and a simple sliding of the anchors on the belts to which they engage to reposition them in the correct locations, a wide variety of specific foundational issues can be addressed for a wide variety of patients. Further, these belt assemblies and elastic members when positioned to anchored mounts, are easily and quickly positioned on the patient which encourages widespread use by therapists and patients alike.

It should be noted, the forgoing examples of related art and limitations related therewith are intended to be illustrative and not exclusive, and they do not imply any limitations on the exercise device and method described and claimed herein. Various limitations of the related art are already known or will become apparent to those skilled in the art upon a reading and understanding of the specification below and the accompanying drawings.

An object of the present invention is the provision of a manual cuing device for foundational issues of multiple patients.

It is another object of the invention to provide such an manual cuing device, which is easily reconfigured by altering band anchor positioning and/or band durometer or resistance, to allow it to be worn by multiple patients to treat multiple foundational issues.

Further objectives of this exercise invention will be brought out in the following parts of the specification wherein the summary and detailed description of the invention are for the purpose of fully disclosing the invention without placing limitations thereon.

### SUMMARY OF THE INVENTION

The present invention provides a solution to the noted shortcomings in the art, through the provision of a device and system which is easily reconfigured to address a wide variety of underlying foundational problems. Once operatively configured to treat one or more patient foundational problems, it continuously provides manual cuing to urge a normal movement pattern.

The device and system herein provided is a multi-configurable elastic band orthotic which is easily customized to treat one or a plurality of such patient foundational problems. Such is accomplished through the changing one or both of the positioning of two or more elastic band anchors, and/or the durometer and resulting resistance of the elastic bands. A plurality of elastic bands can be positioned to impart force along multiple force vectors to a single point on the body of the patient or user to further customize treatment. To that end, the device herein may be employed for a multitude of asymmetries.

For example, the device and system provided herein, has been configured for employment in treatments for foot drop, Trendelenburg gait pattern, crouched gait associated with cerebral palsy, kyphotic posture and gait asymmetries related to Parkinson's Disease, genu valgus related to weak hip external rotators, hemiplegia and recurvatum related to spinal cord injury and multiple sclerosis. Of course the foregoing listing should be considered as an exemplar and not limiting in any fashion. Because of the sliding anchors for the distal ends of the elastic bands, and the varying of the length and the durometer of the elastic bands, a therapist/exercise specialist who has access to this device is only limited by their ability to apply their knowledge kinesiology.

Because of the configuration of wearable belts and slidable anchors and easily connected elastic bands, the device is relatively inexpensive and user friendly. Using cooperative connectors located at the distal ends of the belt assemblies, it can be donned in minutes by the user and modified within seconds. So configured the device may be used as a training tool in a gym just as easily as in a clinic setting. It can also be obtained by or provided to patients, for long term personal use.

In a preferred configuration of the device, in all modes herein, the device includes a chest harness assembly which is adapted for chest and shoulder engagement, and a waist belt assembly configured for removable engagement around the waist of mid section of the user. A user configurable plurality of other components are provided which may include such as one or a plurality of engageable components such as elastic band assemblies, one or more limb strap assemblies, anchors connecting opposing ends of the elastic band which are slidably positionable upon the belt and cord assemblies, shoe insert or bands, and other components noted herein.

The elastic bands can be varied in force by varying the durometer and/or type of material from which they are formed to thereby vary their resistance to elongation during use. Further, the device herein allows for communication of force from such elastic bands, along a plurality of force vectors to a single common engagement point. Thus, the vector of the force as well as a varying of the force of each elastic band may be chosen and connected to a common connection point. This provides substantial utility when aiding or treating a particular physical problem of a user since unequal forces can be communicated at differing vectors, to help move or exercise, or strengthen, or otherwise treat or aid the user.

Additionally provided herein is a hand anchor which consists of two components. A first such component includes a large hand belt which is adapted to wrap around and engage the palm of the hand and wrist. A second component of the hand anchor which is configured to removably engage to the hand belt are one or a plurality of smaller finger wraps. The hand belt section is preferably formed with a hook and loop material on one or both sides which is configured to engage the two when the hand belt is wrapped around the hand. An opening communicating through the body of the hand belt, allows the thumb to communicate therethrough and provide an anchor against sliding when in the wrapped engagement.

There one or plurality of separate finger wraps are preferably made of an elastic material for comfort and a biased engagement of a finger, and has hook or hook and loop material positioned to engage one end of the finger wrap to any position on the hook and loop fabric located on one or both surfaces of the hand belt. The finger wraps when engaged around the finger or fingers and connected to the hand wrap allow the hand to go into a proper position for a better tinodesis grasp.

This hand anchor component works in conjunction with a limb belt through a connection by an elastic band to a bicep-engaged limb belt, to promote flexion or extension of the wrist and or elbow. The hand anchor and limb belt may also be connected to the shoulder complex through the chest harness to facilitate arm swing and placement while ambulating. This system will also cue trunk rotation during the gait cycle. The configuration is such that some persons will need more components of this than others.

With respect to the above description, before explaining at least one preferred embodiment of the herein disclosed Mobility Assisting Apparatus, and system in detail, it is to be understood that the invention is not limited in its application to the details of construction and to the arrangement in the following description or illustrated in the drawings. The mobility assisting invention herein described, is capable of other embodiments and of being practiced and carried out in various ways which will become obvious to those skilled in the art on reading this disclosure. Also, it is to be understood that the phraseology and terminology employed herein are for the purpose of description and should not be regarded as limiting.

As such, those skilled in the art will appreciate that the conception upon which this disclosure is based, may readily be utilized as a basis for designing of other user-worn mobility assisting devices and for carrying out the several purposes of the present disclosed system. It is important, therefore, that the claims herein be regarded, as including such equivalent construction and methodology, insofar as they do not depart from the spirit and scope of the present invention.

### BRIEF DESCRIPTION OF DRAWING FIGURES

FIG. 1 depicts a mode of the device as-used, showing a shoulder harness having vertical straps extending to connections with a first anchor belt shown as a waist belt which is in an encircled engagement of the mid section or waist of the body of the user, with elastic members extending between position-adjustable anchors located on each of a limb strap and the waist belt and between a limb strap on the calf and a foot anchor.

FIG. 2 depicts a rear view of the device herein during engagement with a user to an as-used positioning of the shoulder harness and waist belt providing an anchor belt, and showing the shoulder straps extending criss-crossed through a cross over fastener which particularly preferred to maintain the shoulder straps in proper positioning as well as to provide band connections or anchors through apertures, such as shown in FIG. 5A.

FIG. 3. shows the device in a side view wherein the moveable anchors for the elastic bands are positioned on the anchor belt, which is shown as a waistbelt, and provide for the ability to communicate a first force vector and a second force vector pulling on an anchor point of a limb belt, such as a leg band, and a similar dual vector configuration from a calf band forming the anchor belt, to a toe anchor.

FIG. 4 depicts multiple movable anchors for the elastic bands which have a body with a passage therein allowing adjustable positioning on an anchor belt or limb belt, and also showing the preferred stiffener employable to maintain the passage elongated where flexible fabric or belting material forms the body.

FIG. 5 shows first the device herein with an armband engaged around a bicep area of an arm and forming a first or anchor belt which has multiple anchors for elastic bands extending between moveable anchors on the bicep engaged band and multiple arm and wrist bands.

FIG. 5A shows an enlarged view of the cross over fastener employable as in FIG. 2, which includes a plurality of openings therein for engagement of hooked or other connecters at the distal ends of elastic bands and slots for adjustable positioning of shoulder straps.

FIG. 6 depicts an elastic band having band connectors at opposing ends thereof adapted to connect to band or belt engaged anchors, shown as hooks or carabineers.

FIG. 6A shows another mode of an engageable anchor configured to removably engage with connectors at the distal ends of elastic bands or to provide a mount elastic bands extending in a plurality of force vectors therefrom.

FIG. 7 shows a group of bands or belts adapted to the system herein where D buckles may be positioned at end positions or central positions.

FIG. 8 shows a hand belt formed of hook and loop fabric adapted to positioning around the palm of the hand of a user as in FIG. 9, where a thumb is positioned through an opening therein, along with a slidable anchor having a band mount such as a ring or D buckle, and a finger belt.

FIG. 9 depicts the hand anchor component of the device which includes a hand belt of FIG. 8, operatively engaged around the palm and rear of the hand of a user, and showing the one or plurality of the elastic finger belts engaged at one or opposing ends to the surface of the hand belt, as well as a first anchor belt provided by a bicep-positioned limb band providing a mount for the opposing end of the elastic band.

### DETAILED DESCRIPTION OF THE PREFERRED EMBODIMENTS OF THE INVENTION

In this description, any directional prepositions if employed, such as up, upwardly, down, downwardly, front, back, first, second, top, upper, bottom, lower, left, right and other such terms refer to the device or depictions as such may be oriented are describing such as it appears in the drawings and are used for convenience only. Such terms of direction and location are not intended to be limiting or to imply that the device herein has to be used or positioned in any particular orientation. Further, where the term “substantially” is used, unless defined otherwise, it means plus or minus five percent.

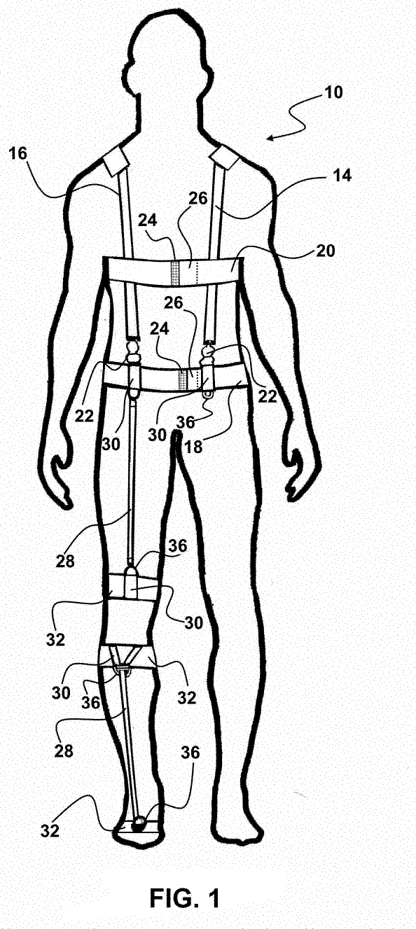

Now referring to drawings in FIGS. 1-9, wherein similar components are identified by like reference numerals, there is seen in FIG. 1 one preferred mode of the device 10. In FIG. 1, is shown the device 10 in an as-used configuration upon a user. The device 10 in one or more modes features a shoulder harness 12 which includes at least a first shoulder strap 14 and a second shoulder strap 16 which engaged with a waist belt 18. In all modes, the device 10 features an anchor belt which has one or more anchors 30 upon it which engage with one end of an elastic band which extends to a limb belt 32 which as shown herein may be located on the leg or foot or arm of the user depending on the treatment or movement aid desired. As noted below, it is preferred that the anchors 30 are slidably locatable to different locations to provide adjustment of force vectors or angles.

As shown in FIG. 1, the anchor belt is provided by a waist belt 18 and preferably is adjustable for size, to accommodate different sized users. Such may be accomplished by the provision of an adjustable fastener 24 such as hook and loop fabric, and an overlap 26 of a portion of the waist belt 18 adjacent the adjustable fastener 24. This allows the user to cinch the waist belt 18 tight and engage the adjustable fasteners 24 once done.

Additionally shown in FIG. 1, which includes the shoulder harness in the device, is a frontal engagement of the two straps 14 and 16 of the shoulder harness 12 which engage to form a torso strap 20. This engagement of the two straps has been found to make it easier for the user to don the shoulder harness 12 and to help maintain the first shoulder strap 14 and second shoulder strap 16 in fixed spacing from each other on the front of the user. Engagement of these two ends forming the torso strap is also preferably adjustable using the adjustable fastener 24 and overlap 26 area, to allow for the encircling size to be adjusted to each user.

As can be seen in FIG. 1, a strap connector 22 engages the first shoulder strap 14 and second shoulder strap 16 to the waist belt 18. While this could be a fixed engagement where the strap connector 22 is provided by sewing or riveting the two shoulder straps 14 and 16 to connect them to the waist belt 18, Currently, a strap connector 22 formed of two halves which removable mate together, is preferred for ease of use. Such may be snaps, clips, or other complementary mating fasteners where one half of the strap connector 22 is positioned at the distal end of the shoulder strap 14 or 16, and the other half of the strap connector 22 is engaged to the waist belt 18.

Additionally shown in FIG. 1, and in other figures herein, are elastic bands 28 which, as noted, may be adjusted for resistive force, by a change in the diameter or durometer thereof and changing the material forming the elastic bands 28. For example, rubber forming the elastic bands 28 has a differing resistance to elongation than does polyethylene. Also, forming the elastic bands 28 in differing unstretched lengths will vary the resistive force once secured at opposing ends. Further, forming the elastic bands 28 hollow will impart a differing resistance to elongation and a force to the opposing connection points, than an elastic band 28 formed with a solid core. In all modes of the device 10 herein, it is preferred that the elastic bands 28 be provided or available from a kit or group of such elastic bands 28, where each has a different resistive force once in an elongated position where opposing ends of the elastic band 28 are connected between two anchors 30.

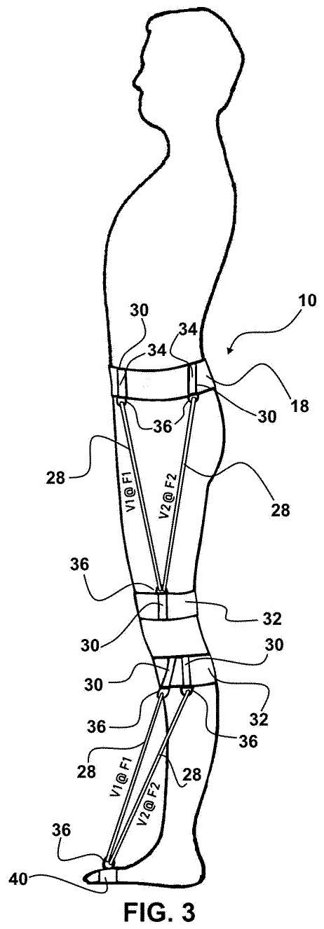

Also, particularly preferred in all modes of the device 10 herein, are anchors 30 which are not fixed but are slidably positionable to any point on the waist belt 18 and/or any of the limb belts 32. This sliding positionable engagement is preferred because it allows the user or the medical professional to position the anchors 30 perpendicular and inline or at chosen angles to each other thereby adjusting the vector of the force imparted to the anchors 30 positioned on limb belts 32. Such is shown for example in FIG. 3, where elastic bands 28 have opposing ends engaged to and extending from the slide-positioned anchors 30, at chosen vectors and force.

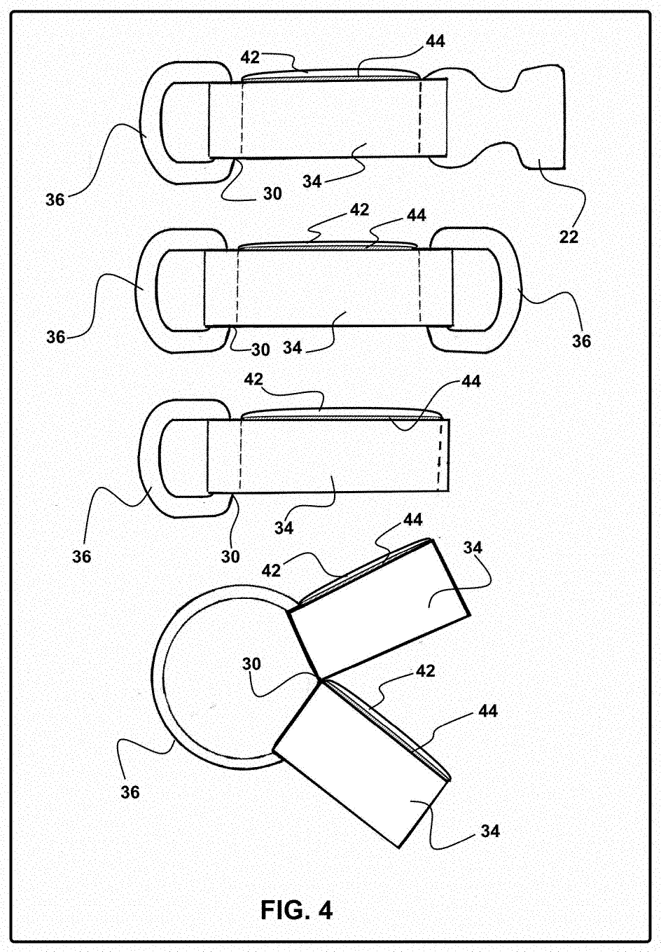

The anchors 30 are formed with an anchor body 34 which has a band mount 36 positioned thereon. Such is described below in FIG. 4 which shows a plurality of different anchors 30 having a body 34 adapted to slide to a chosen position along the length of the waist belt 18 or the limb belts 32, and have band mounts 36 configured to removably engage to one of the two ends of the elastic bands 28.

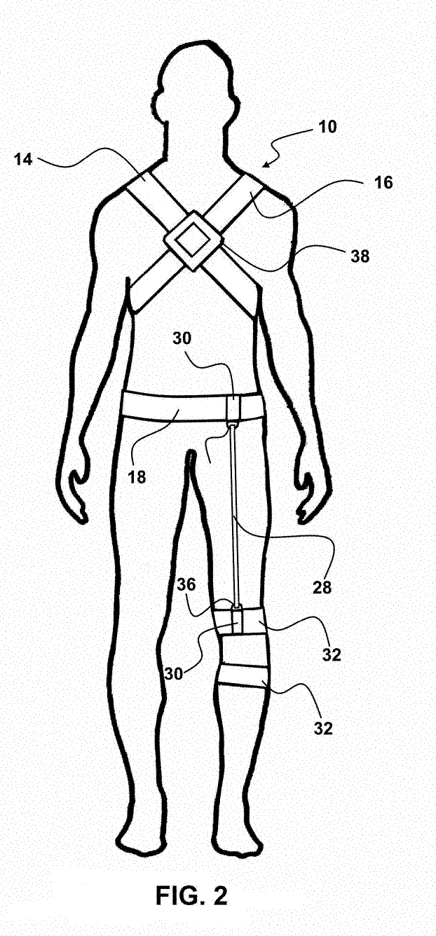

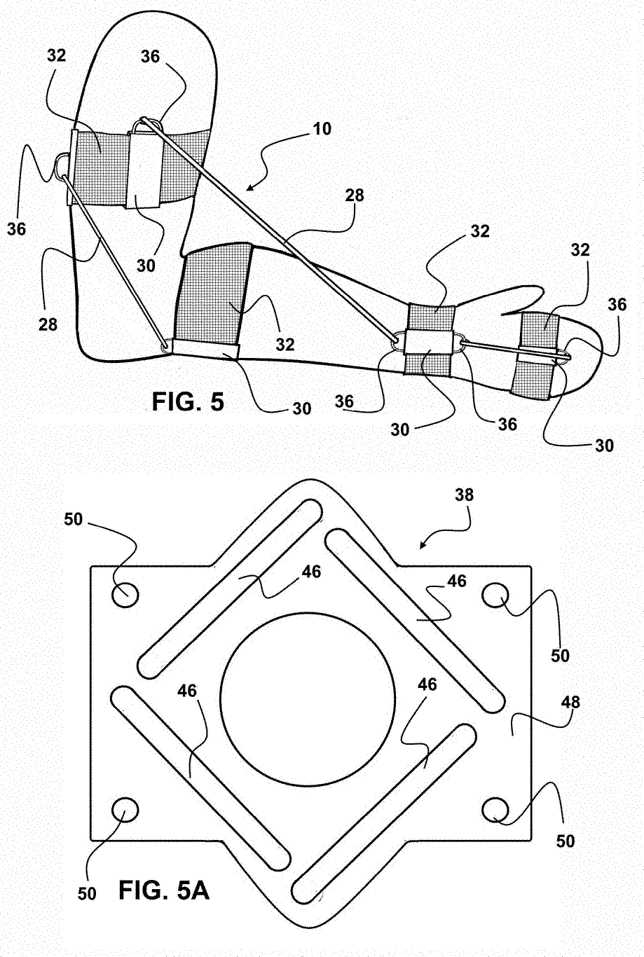

Depicted in FIG. 2 is a rear view of the device 10 in the as-used position on a user. Shown are the first shoulder strap 14 and second shoulder strap 16, extending in a criss-crossed positioning. Such has been found to better support the harness 12 on the user during use. While sewing or permanent fastening of the two straps can be used to hold them in a criss-crossed position, the device 10 herein provides a cross over fastener 38 which has slots or other adjustable engagements to hold the first shoulder strap 14 and second shoulder strap 16 in a crossed positioning, but allows for linear adjustment of that positioning with separate pathways for each strap which can be better seen in FIG. 5A.

In FIG. 3 is depicted the device 10 in the as-used positioning on a user, showing the function of positioning the anchors 30 at any chosen position along the length of the waist belt 18 or the limb belts 32, to provide force at a chosen angle or vector, to a band mount 36 engaged to an anchor body 34. As shown, one or preferably a plurality such as two elastic bands 28 can connect at respective first ends to a common band mount 36 on the anchor body 34 of an anchor 30 which has been slid to a chosen position on a limb belt 32.

As shown in FIG. 3, a first elastic band 28 extending between an engagement with a first anchor 30 slid to position on the waist belt 18, imparts a force to a common band mount 36 on the anchor body 34 slid to position on the limb belt 32. As shown this first force imparted is along a first vector V! And a first force F1. A second elastic band 28 is positioned using the moveable anchors 30, to impart a second force at a second vector or angle, which is shown as V2 at force F2. Thus, using the position-adjustable anchors 30 and any of a kit of different elastic bands 28 having differing elongation resistance, the user can impart equal or unequal forces to the single point at the anchor 30 shown located on the limb belts 32. This same adjustable force along two or more vectors is shown being provided by the two elastic bands 28 extending between connections to band mounts 36 on anchors 30 positioned on a limb belt 32, and a band mount 36 connected to a foot band 40. The foot band 40 may also be a shoe or stiff sock or may just be a foot band engaged therearound.

As noted, FIG. 4, depicts multiple slide-positionable anchors 30 which may be employed in the device 10 herein. Of course such should not be considered limiting.

As shown, the anchor body 34 of each anchor 30, has a passage 42 communicating through the anchor body 34. This passage 42 is sized to allow for a sliding engagement upon the waist belt 18 or a limb belt 32 or the like, to allow the user to locate the anchors 30 in appropriate positions on the waist belt 18 and/or the limb belts 32, to achieve the force from an elastic band 28 upon an anchor 30, at the desired angle or vector and at the desired magnitude. As noted, this sliding adjustable engagement allows for multiple elastic bands 28 to be employed to achieve the desired force F, at the desired vector V, as shown in FIG. 3.

The anchor body 34 may be formed of plastic or metal or other materials in a manner to yield the passage 42 therethrough. Currently, the preferred mode of the device 10 forms the anchor body 34 from woven or non woven textile fabric such as nylon or polyester belting. To maintain the passage 42 open and elongated, a stiffener 44 is positioned along one or both sides of the passage 42. This stiffener 44 may be flexible but will maintain its length and will not fold, and thereby holds the passage 42 to the proper size. The stiffener may be formed of metal or polymeric materials such as nylon or polyester or the like.

FIG. 5 shows first the device 10 herein with a limb belt 32 forming an armband engaged around a bicep area of an arm. This bicep-engaged limb belt 32 has a plurality of anchors 30 located in appropriate positions thereon to locate multiple band mounts 36 to which one end of respective elastic bands 28 are connected. As shown, one elastic band 28 is elongated and engaged between connections to a band mount 36 on an anchor 30 on the limb belt 32 on the bicep, and a band mount 36 on an anchor 30 slid to position on an arm-positioned limb belt 32. A second elastic band 28 extends from a second band mount 36 located on a second anchor 30 positioned on the limb belt 32 on the bicep and a band mount 36 connected to an anchor 30 located on a writs-positioned limb belt 32.

As also can be seen, on an opposite side of the anchor 30 located on the limb belt 32 at the wrist, a third elastic band 28 is connected between a band mount 36 on the wrist-positioned limb belt 32, and a band mount 36 on the anchor 30 located on a limb belt 32 encircling the hand of the user. This allows the multiple elastic bands 28 to impart determined resistance force along multiple vectors, to achieve the desired result. FIG. 5 is of course exemplar of the adjustability of the device 10 herein, and in no way limiting.

As noted, a cross over fastener 38 is desirable to maintain the first shoulder strap 14 in a criss-crossed position with the second shoulder strap 16, on the back of the user as shown in FIG. 2. However, it is important that the straps be adjustable in their positioning and engagement with the cross over fastener 38 to allow for adjusting the device 10.

As shown, a plurality of diagonal slots 46 communicate through the fastener body 48 and run in parallel pairs to allow for threading of the first and second straps through the appropriate pair of parallel diagonal slots 46. Additionally provided in the cross over fastener 38, are a plurality of openings 50 communicating through the fastener body 48. These openings 50 may be employed to secure one end of an elastic band 28 to provide the band mount 36 for such.

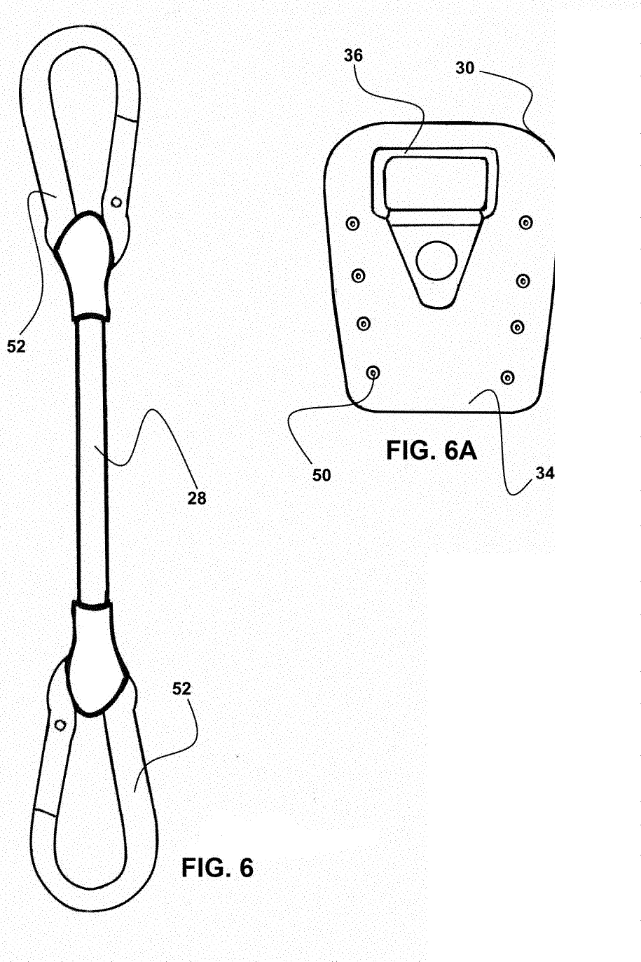

In FIG. 6 is shown, an example of an elastic band 28 having band connectors 52 connected to opposing ends of the elastic bands 28. Shown are hooks which have a spring loaded closure. However, hooks which are open or snaps or rings or other forms of band connectors 52 as would occur to those skilled in the art, such as available in the GRAINGER catalog, are anticipated within the scope of this patent. Additionally, while not as convenient, one or both of the opposing ends of the elastic bands 28 could be tied onto the band mount 36 and the knot achieved would provide the band connector 52.

Shown in FIG. 6A, is another mode of an engageable anchor 30 where the band mount 36 is engaged against a side surface of the anchor body 34. This mode has the same passage 42 for slidable locating of the anchor 30 to appropriate positions on the waist band 18 or the limb belts 32.

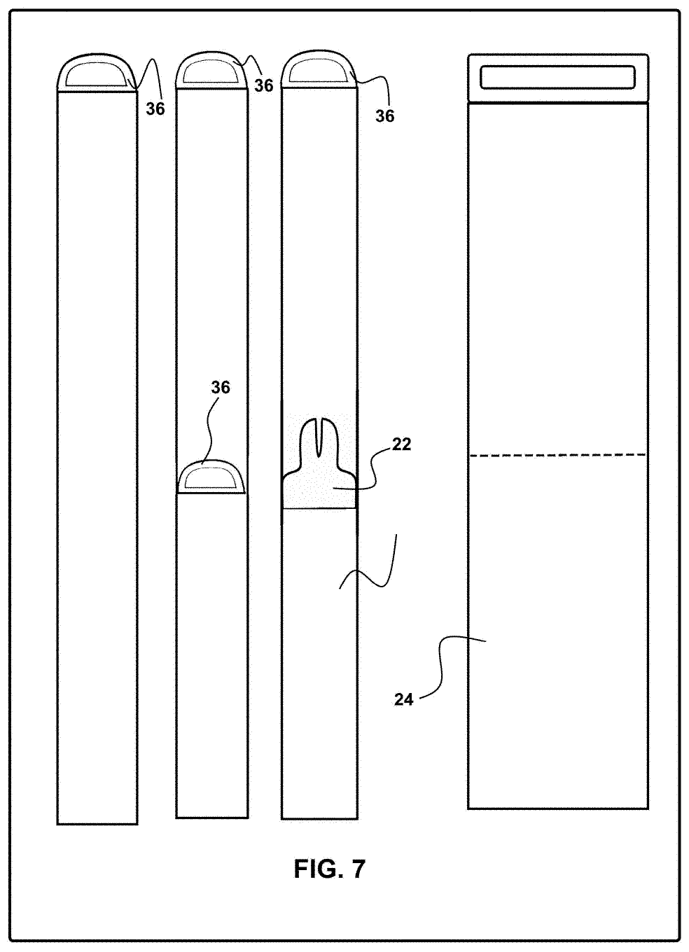

In FIG. 7, while not to scale, is depicted a group of band or limb belts 32 adapted to the system herein. The limb belts 32 will be adjustable in circumference when secured around the limb of the user, through the employment of an adjustable fastener such as hook and loop fabric 24 shown in FIG. 1. As depicted, the band mounts 36 are formed by D style buckles but could also be rings, hooks, or other mounts as would occur to those in the art. Additionally shown is a waist belt 18 extension which would engage with the adjustable fastener 24 shown in FIG. 1.

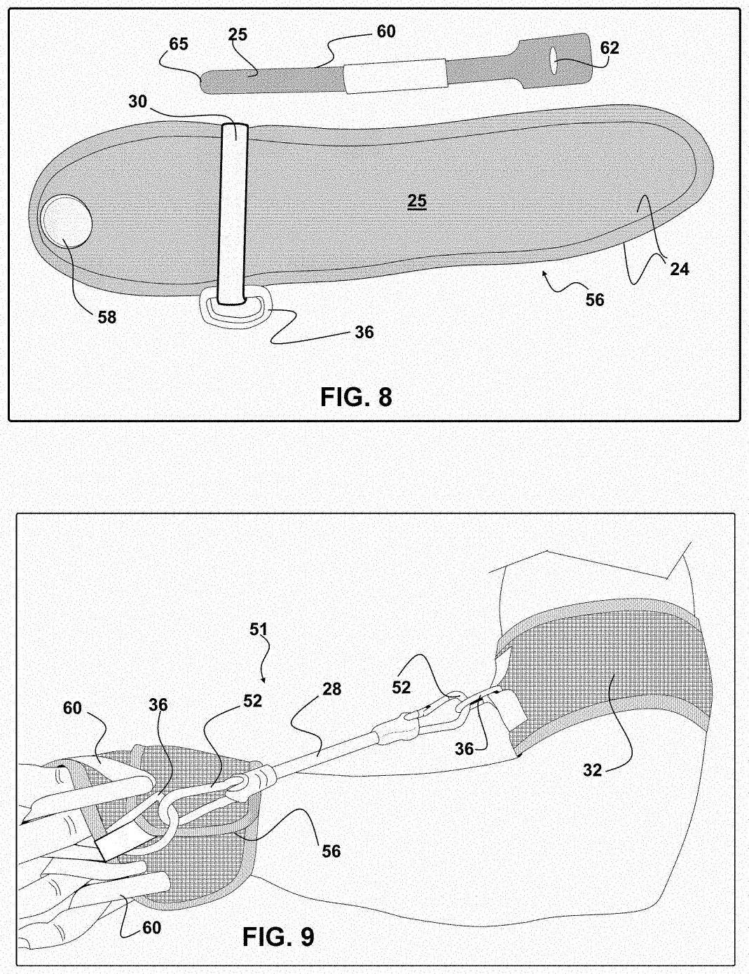

Shown in FIG. 8, is a limb belt which is especially preferred and provided in the form of a hand belt 56 which is connected via an elastic band 28 to an anchor belt which as shown in FIG. 9, is provided by a limb belt 30 positioned on the bicep. The hand belt 56 includes the anchor 30 with a passage 42 adapted to slide upon the body of the hand belt 56. Especially preferred in the hand belt 56 is an aperture 58 communicating through the body of the hand belt 56 which is adapted for communication of a thumb of the user therethrough whereupon the hand belt 56 is wrapped around the hand as shown in FIG. 9. This provides an especially secure engagement of the hand belt 56 to keep it from slipping toward the wrist of the user.

The hand belt 56 is formed with a surface of hook and loop type fabric 25 which is formed with both hook and loop portions on a single surface. Alternatively, the hook portion may be formed on one surface and the loop on the opposing surface, however the combination hook and loop fabric 25 is preferred as it is easier for use, and, is adapted to connect to the finger belt 60 easier as shown in FIG. 9.

In FIG. 9, as noted, is shown the hand belt 36 in operative engagement as-used where it has been wrapped around the hand and palm of the user, with their thumb positioned through the aperture 58 shown in FIG. 8. Also shown is a limb belt 32 engaged around the bicep. The limb belts 32, in all modes of the device 10, may be of fixed circumference and preferably formed of elastic fabric such that they will slide upon the limb and contract in a biased engagement to provide a more secure non-sliding engagement.

Alternatively, the limb belts 32 can be formed similar to the waist belt 18 and torso strap 20 and have an adjustable fastener 24 such as hook and loop fabric and an overlapping portion 26 as shown in FIG. 1, to allow the user to encircle the limb of choice and engage the adjustable fastener 24 on both to form a snug or biased contracting fit.

As shown in FIG. 9 the hand anchor component 51 includes a band mount 36 engaged to the limb belt 32. The preferably elastic limb belt 32 is positioned to a biased compressive engagement of the bicep. As shown the limb belt 32 provides an engagement for the band connectors 52 which are shown as hooks with spring loaded hinged members but which as noted herein, can be other snaps, hooks, or the like as would occur to those skilled in the art. The band mount 36 is shown in fixed positioning but can be formed as a slidable anchor 30 such as shown on the hand belt 56 in FIG. 8 should the function of adjusting the vector or angle of the force provided by the elastic band 28 be required for a specific treatment.

The elastic band 28 as shown in FIG. 9, extends to a second end where it is engaged to the band mount 36 connected to the sliding anchor 30 on the hand belt 56 to provide adjustability of the force vector of the elastic band 28. The elastic band 28 so engaged imparts the resistance force provided by the chosen elastic band 28 at the chosen vector or angle, to movement of the hand and forearm, and as noted below, to hold the fingers more open when the hand is supposed to be relaxed.

Additionally shown, as noted, are the preferably elastic finger belts 60 shown in full form in FIG. 8 and in operative engagement in FIG. 9. The finger belts 60 shown in FIG. 8 may have a slit 62 at one end thereof through which the second end 65 of the finger belt 60 may be threaded to form an encircling biased engagement of a finger with the elastic material of the finger belts 60. At least a portion of one side surface of the finger belt 60, has hook and loop fabric 25 thereon as an adjustable fastener 24. Of course the entire surface or opposing surfaces may be covered. However, the hook and loop fabric employed must be soft to the touch for a comfortable finger engagement.

In use as shown in FIG. 9, the finger belt 60 may be either looped around the palm side of the finger and have the adjustable fastener 24 thereon engaged to a cooperative engaging surface of the hand belt 56, using hook and loop fabric on a surface of the finger belt 60. Alternatively, the second end 65 of the finger belt 60 may be threaded through the slit 62, and the finger encircled by the loop formed in the finger belt 60.

In either mode of use, the finger belt 60, is then engaged to the surface of the hand belt 56 using an adjustable fastener such as hook and loop fabric 25. Once removably engaged to the mating surface of the hand belt 56, the finger belt 60 allows the hand to go into a position for better tinodesis grasp. Further, the hand anchor 51 so configured with the hand belt 56 connected by the elastic band 28 to the limb belt promotes flexion or extension of the wrist and or elbow. As noted above, the hand anchor 51 may be connected to the shoulder complex through the chest harness to facilitate arm swing and placement while ambulating or to cue trunk rotation during the gait cycle.

While all of the fundamental characteristics and features of the mobility assisting apparatus and system herein have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that upon reading this disclosure and becoming aware of the disclosed novel and useful device and system herein disclosed, that various substitutions, modifications, and variations may occur to and be made by those skilled in the art, without departing from the spirit or scope of the invention. Consequently, all such modifications and variations and substitutions, as would occur to those skilled in the art are considered included within the scope of the invention as defined by the following claims.

What is claimed is:

1. A mobility assisting apparatus, comprising:
an anchor belt engageable to an as-used position encircling a portion of the waist or a limb of the body of a user;
a first anchor positioned on said anchor belt;
a limb belt positioned on a portion of an arm or leg of said user;
said limb belt having a second anchor thereon;
a first elastic band having a first end thereof in a first connection with said first anchor, said first elastic band having a second end thereof in a second connection with said second anchor;
said first elastic band imparting a contracting biasing force along a force vector running at an angle between said first connection and said second connection.

2. The mobility assisting apparatus, of claim 1 additionally comprising:
said first anchor having a body, said body of said first anchor having a first passage therethrough, said first passage in a sliding engagement upon said anchor belt;
said second anchor having a body, said body of said second anchor having a second passage therethrough in a sliding engagement with said limb belt;
a first sliding adjustment of said first anchor positioning said first anchor to any of multiple locations on said waist belt;
a second sliding adjustment of said second anchor positioning said second anchor to any of multiple locations on said waist limb belt; and
said angle of said force vector being adjustable by either of said first sliding adjustment or said second sliding adjustment.

3. The mobility assisting apparatus, of claim 2 additionally comprising:
one or both of said body of said first anchor and said body of said second being formed of flexible material; and
stiffeners positioned adjacent said first passage of said first body and said second passage when formed of said flexible material.

4. The mobility assisting apparatus, of claim 2 additionally comprising:
said anchor belt being a waistbelt configured for removable engagement around a waist of a user.

5. The mobility assisting apparatus, of claim 3 additionally comprising:
said anchor belt being a waistbelt configured for removable engagement around a waist of a user.

6. The mobility assisting apparatus, of claim 4 additionally comprising:
a shoulder harness adapted to engage a user in an as-used position;
said shoulder harness having a first shoulder strap extending between a first end and a second end;
said shoulder harness having a second shoulder strap extending between a first end and a second end;
said second end of said first shoulder strap in a removable connection to said first anchor in said sliding engagement upon said waist belt; and
said second end of said second shoulder strap in a second removable connection to a secondary anchor in a sliding engagement upon said waist belt.

7. The mobility assisting apparatus, of claim 5 additionally comprising:
a shoulder harness adapted to engage a user in an as-used position;
said shoulder harness having a first shoulder strap extending between a first end and a second end;
said shoulder harness having a second shoulder strap extending between a first end and a second end;
said second end of said first shoulder strap in a removable connection to said first anchor in said sliding engagement upon said waist belt; and
said second end of said second shoulder strap in a second removable connection to a secondary anchor in a sliding engagement upon said waist belt.

8. The mobility assisting apparatus, of claim 6 additionally comprising:
said first end of said first shoulder strap having a removable engagement with said first end of said second shoulder strap to form a torso strap extending across a waist area of said user;
said first shoulder strap and said second shoulder strap extending from said removable engagement forming said torso strap, to a criss-cross positioning therebetween, adjacent the back of said user with said harness in said as-used position;
said first shoulder strap extending from said criss-cross engagement and over a first shoulder of said user to said removable connection with said first anchor; and
said second shoulder strap extending from said criss-cross engagement and over a second shoulder of said user to said second removable connection with said secondary anchor.

9. The mobility assisting apparatus, of claim 7 additionally comprising:
said first end of said first shoulder strap having a removable engagement with said first end of said second shoulder strap to form a torso strap extending across a waist area of said user;
said first shoulder strap and said second shoulder strap extending from said removable engagement forming said torso strap, to a criss-cross positioning therebetween, adjacent the back of said user with said harness in said as-used position;
said first shoulder strap extending from said criss-cross engagement and over a first shoulder of said user to said removable connection with said first anchor; and
said second shoulder strap extending from said criss-cross engagement and over a second shoulder of said user to said second removable connection with said secondary anchor.

10. The mobility assisting apparatus, of claim 8 additionally comprising:
said criss-cross positioning being through respective pairs of diagonal slots located in a fastener body.

11. The mobility assisting apparatus, of claim 9 additionally comprising:
said criss-cross positioning being through respective pairs of diagonal slots located in a fastener body.

12. The mobility assisting apparatus, of claim 2 additionally comprising:
said anchor belt configured for operative positioning around a bicep of said user;
said limb belt defining a hand belt having an opening therein configured for engagement with a thumb of said user; and
said hand belt configured for a wrapped engagement around a hand of said user with said thumb engaged in said opening.

13. The mobility assisting apparatus, of claim 12 additionally comprising:
one or a plurality of elastic finger belts adapted to encircle a finger of said user at a first end and to removably engage with said hand belt at a second end.

14. The mobility assisting apparatus, of claim 3 additionally comprising:
said anchor belt configured for operative positioning around a bicep of said user;
said limb belt having an opening therein configured for engagement with a thumb of said user; and
said limb belt configured for a wrapped engagement around a hand of said user with said thumb engaged in said opening.

15. The mobility assisting apparatus, of claim 2 additionally comprising:
a second elastic band having a first end thereof in a connection with a secondary anchor slidably located upon said anchor belt;
a second end of said second elastic band engaged to said second anchor;
said first elastic band imparting a contracting biasing force along a first force vector running at a first angle between said first connection and said second connection;
said second elastic band imparting a contracting biasing force along a second force vector running at a second angle between said secondary anchor on said anchor belt and said second anchor on said limb belt; and
one or both of said first angle and said second angle being adjustable by a sliding of any of said first anchor or said second anchor or said secondary anchor in their respective sliding engagements.

16. The mobility assisting apparatus, of claim 3 additionally comprising:
a second elastic band having a first end thereof in a connection with a secondary anchor slidably located upon said anchor belt;
a second end of said second elastic band engaged to said second anchor;
said first elastic band imparting a contracting biasing force along a first force vector running at a first angle between said first connection and said second connection;
said second elastic band imparting a contracting biasing force along a second force vector running at a second angle between said secondary anchor on said anchor belt and said second anchor on said limb belt; and
one or both of said first angle and said second angle being adjustable by a sliding of any of said first anchor or said second anchor or said secondary anchor in their respective sliding engagements.
